(19) United States
(12) Patent Application Publication
(10) Pub. No.: US 2012/0136205 A1
(43) Pub. Date: May 31, 2012

(54) DILDO FOR SEXUAL STIMULATION

(75) Inventor: Jose Antonio Aguilera Galeote, Campanillas-Malaga (ES)

(73) Assignee: INNOVACIONES SOSTENIBLES DEL SUR, S.L.U., Campanillas-Malaga (ES)

(21) Appl. No.: 13/322,727

(22) PCT Filed: May 28, 2010
(86) PCT No.: PCT/ES2010/070358

§ 371 Date: Feb. 14, 2012

(30) Foreign Application Priority Data
May 29, 2009 (ES) P200930230

(51) Int. Cl. A61F 5/00; A61H 1/00

(52) U.S. Cl. 600/38; 601/46

(57) ABSTRACT

The object of the invention relates to a dildo for sexual stimulation, comprising a tubular body (1) with a hollow structure made of a smooth material, such as silicone or rubber, having a shape and size similar to a penis, filled with a non-Newtonian fluid (2), the viscosity of which increases as the tension gradient applied thereto increases.

### OBJECT OF THE INVENTION

The present invention relates to an implement used for sexual stimulation, especially by women. It is therefore an instrument comprising a tubular body with a hollow structure made of a smooth material, such as silicone or rubber, having a shape and size similar to a penis.

### BACKGROUND OF THE INVENTION

The dildo is a sexual toy used from time immemorial for masturbation both for men and women, there being signs that it was used some 3,000 years ago. In some sexual relationships it is also common for it to be added as part of a couple's sexual play.

Currently there are dildos of all sizes, shapes, and flexibilities, such that they can reach the main erogenous zones of the body, flavors and colors for those who do not like the taste or texture of the element it is made from, etc.

A more current and improved variant of the dildos would be vibrators, devices or implements combining a dildo with a vibration mechanism facilitating sexual stimulus, particularly for women.

Currently it is generally accepted that the use of dildos and vibrators does not cause any negative sexual effect and that they can also be enjoyed by couples, as a part of a healthy sexual relationship.

Despite the known multiple variants of these instruments, all of them have a rigid or flexible body, but of a non-modifiable rigidity, which clearly constitutes a drawback when simulating the situation of the penis during sex, which changes in the degree of erection and therefore of “rigidity” depending on the moment of the act.

Document DE 299 21 064 U1 describes a soft vibrator, internally comprising a liquid (sodium acetate) which hardens and heats up by means of a chemical reaction triggered at a given time. The reaction is started by folding a metal strip which is floating within said liquid (hot ice). Its operation is equivalent to a very popular hand warmer in Nordic countries. When the material has acquired a certain degree of hardness, it does not return to being soft on its own and in no case does its degree of hardness depend on the speed of the vibrator.

### DESCRIPTION OF THE INVENTION

The most notable feature of the dildo of the invention is that it changes texture or degree of rigidity depending on the desires of the user by simply modifying the pressure or speed to which the device is subjected, or also depending on the activation of an internal vibrator. The device returns to its initial state, soft and/or flaccid, when the user turns off the motor of the vibrator, or when the movement pressure or speed of the device ceases, such that the degree of hardness of the vibrator can be regulated through the latter.

This novel effect is achieved with a hollow, flexible body which is filled with a non-Newtonian fluid. Such fluids have a performance and features which are clearly advantageous compared to traditional fluids when they are subjected to different pressures given that their viscosity varies with the pressure gradient applied thereto. As a result, a non-Newtonian fluid does not have a defined and constant viscosity value, unlike a Newtonian fluid. These fluids can therefore be better characterized by means of other rheological properties, properties having to do with the relationship between stress and tensions under different flow conditions, such as shear or oscillatory stress conditions. A non-Newtonian fluid subjected for example to an impact makes the fluid behave more like a solid than a liquid, however if pressure is slowly applied on the non-Newtonian fluid its behavior seems more like a liquid than a solid since its viscosity has considerably reduced.

Thus the non-Newtonian fluid itself acts as control means for the resistance to deformation of the dildo, depending on the speed and pressure to which it is subjected.

The non-Newtonian material used is a “dilatant” fluid made up of a concentrated solution of starch or sugars in water, or another similar fluid which is not harmful for the human body.

It has also been provided to include a vibrating mechanism in the body of the dildo, within the non-Newtonian material itself, with which the latter mechanism interacts increasing its viscosity and therefore the rigidity of the member when it is put to use.

### DESCRIPTION OF THE DRAWINGS

To complement the description being made and for the purpose of aiding understanding of the features of the invention, a set of drawing is attached to the present specification in which the following has been depicted with an illustrative and non-limiting character:

FIG. 1 is a partially sectioned general elevation view of a dildo made according to the invention.

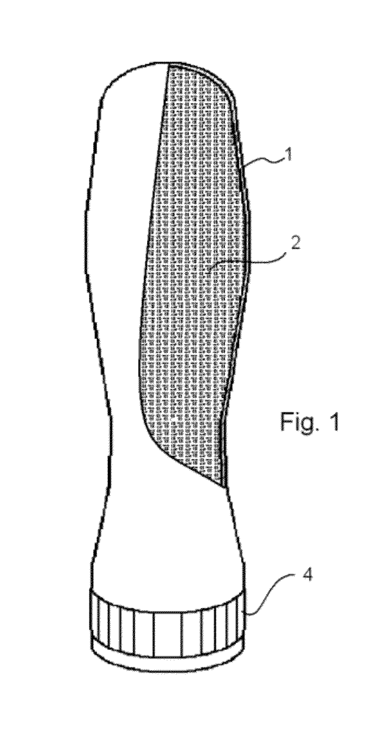

FIG. 2 shows a dildo similar to the preceding one, in this case provided with a vibrating mechanism.

### PREFERRED EMBODIMENT OF THE INVENTION

As can be observed in the mentioned drawings, the dildo of the invention comprises a hollow body (1) made of a flexible and soft material, such as silicone or rubber, and it defines an elongated cylindrical element, a plug (4) preferably being provided at its base. The inside of this body is filled with a non-Newtonian fluid (2) taking up the entire interior space keeping it flexible but without the existence of non-fluid filled spaces.

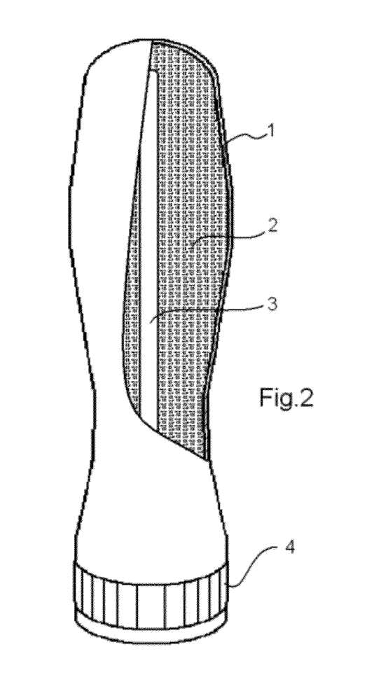

A similar dildo is observed in the option depicted in FIG. 2, which includes a vibrating mechanism (3) arranged therein, the batteries and the vibrating device being hidden in the area of the plug (4).

The operation of this dildo is initially like a conventional flexible-type and, up to a certain point, flaccid dildo. Since it is filled with a non-Newtonian fluid, as it is subjected to greater pressure, either from grabbing it by hand or by that which the vibrating mechanism (3) exerts on the fluid, this fluid becomes increasingly more rigid until reaching practically the texture and rigidity of a solid.

Having sufficiently described the nature of the invention as well as a preferred embodiment thereof, it is stated for all intents and purposes that the materials, shape, size and arrangement of the elements described may be modified provided this does not entail altering the essential features of the invention which are claimed below:

1. A dildo for sexual stimulation, comprising a tubular body (1) with a hollow structure made of a smooth material, such as silicone or rubber, having a shape and size similar to a penis, characterized in that said body is filled with a non-Newtonian fluid (2), the viscosity of which increases as the tension gradient applied thereto increases, such that the fluid itself acts as a control means for the rigidity which the dildo provides against deformation, depending on the speed and pressure to which it is subjected during use.

2. The dildo according to claim 1, characterized in that the non-Newtonian material (2) used is a “dilatant” fluid, made up of a concentrated solution of starch or sugars in water, or another similar fluid which is not harmful for the human body.

3. The dildo according to claim 1, characterized in that it incorporates a vibrating mechanism (3) included in the non-Newtonian material (2) with which it interacts increasing its viscosity and therefore the rigidity of the member when it is put to use.
